SEQUENCE LISTING (19 sequences)

SEQ ID NO: 1 - protein, 115 aa, synthetic construct
EVQLVESGGGLVQPGGSLRLSCAASGFTFSTNAMSWVRQAPGKGLEWVSRLRPKSDNYATYYADSVKGRFTISRDNSKNTLYLQMNSLRAEDTAVYYCAKGTGFWGQGTTVTVSS

SEQ ID NO: 2 - protein, 115 aa, synthetic construct
EVQLVESGGGLVQPGGSLRLSCAASGFTFSTNAMSWVRQAPGKGLEWVARLRPKSDNYATYYADSVKGRFTISRDNSKNTLYLQMNSLRAEDTAVYYCVTGTGFWGQGTTLTVSS

SEQ ID NO: 3 - protein, 115 aa, synthetic construct
EVQLVESGGGLVQPGGSLRLSCAASGFTFSTNAMSWVRQAPGKGLEWVARLRPKSDNYATYYADSVKGRFTISRDDSKNTLYLQMNSLRAEDTAVYYCVTGTGFWGQGTTLTVSS

SEQ ID NO: 4 - protein, 115 aa, synthetic construct
EVQLVESGGGLVQPGGSLRLSCAASGFTFSTNAMSWVRQAPGKGLEWVARLRPKSDNYATYYADSVKGRFTISRDDSTSTLYLQMNSLRAEDTAVYYCVTGTGFWGQGTTLTVSS

SEQ ID NO: 5 - protein, 113 aa, synthetic construct
DIVMTQSPDSLAVSLGERATINCKSSQSLLNSGNQKNYLTWYQQKPGQPPKLLIYWTSTRESGVPDRFSGSGSGTDFTLTISSLQAEDVAVYYCQNDYTSPYTFGQGTKLEIK

SEQ ID NO: 6 - protein, 113 aa, synthetic construct
DIVMTQSPDSLAVSLGERVTMNCKSSQSLLNSGNQKNYLTWYQQKPGQPPKLLIYWTSTRESGVPDRFSGSGSGTDFTLTISSVQAEDVAVYYCQNDYTSPYTFGQGTKLEIK

SEQ ID NO: 7 - protein, 113 aa, synthetic construct
DIVMTQSPDSLAVSLGERATINCKSSQSLLNSGNQKNYLTWYQQKPGQPPKLLFYWTSTRESGVPDRFSGSGSGTDFTLTISSLQAEDVAVYYCQNDYTSPYTFGQGTKLEIK

SEQ ID NO: 8 - protein, 113 aa, synthetic construct
DIVMTQSPDSLAVSLGERVTMNCKSSQSLLNSGNQKNYLTWYQQKPGQPPKLLFYWTSTRESGVPDRFSGSGSGTDFTLTISSVQAEDVAVYYCQNDYTSPYTFGQGTKLEIK

SEQ ID NO: 9 - protein, 113 aa, synthetic construct
DIVMTQSPDSLAVSLGERVTMNCKSSQSLLNSGNQKNYLTWYQQKPGQPPKLLFYWTSTRESGVPDRFSGSGSGTDFTLTISSVQAEDLAVYYCQNDYTSPYTFGQGTKLEIK

SEQ ID NO: 10 - protein, 113 aa, synthetic construct
DIVMTQSPDSLAVSLGERVTMNCKSSQSLLQSGNQKNYLTWYQQKPGQPPKLLFYWTSTRESGVPDRFSGSGSGTDFTLTISSVQAEDLAVYYCQNDYTSPYTFGQGTKLEIK

SEQ ID NO: 11 - protein, 113 aa, synthetic construct
DIVMTQSPDSLAVSLGERVTMNCKSSQSLLSSGNQKNYLTWYQQKPGQPPKLLFYWTSTRESGVPDRFSGSGSGTDFTLTISSVQAEDLAVYYCQNDYTSPYTFGQGTKLEIK

SEQ ID NO: 12 - protein, 10 aa, synthetic construct
GFTFSTNAMS

SEQ ID NO: 13 - protein, 12 aa, synthetic construct
RLRPKSDNYATY

SEQ ID NO: 14 - protein, 6 aa, synthetic construct
VTGTGF

SEQ ID NO: 15 - protein, 17 aa, synthetic construct
KSSQSLLNSGNQKNYLT

SEQ ID NO: 16 - protein, 17 aa, synthetic construct
KSSQSLLQSGNQKNYLT

SEQ ID NO: 17 - protein, 17 aa, synthetic construct
KSSQSLLSSGNQKNYLT

SEQ ID NO: 18 - protein, 7 aa, synthetic construct
WTSTRES

SEQ ID NO: 19 - protein, 9 aa, synthetic construct
QNDYTSPYT

(19) United States
(12) Patent Application Publication
(10) Pub. No.: US 2026/0174847 A1
(43) Pub. Date: Jun. 25, 2026

(54) METHODS OF TREATING DIGESTIVE DISEASES AND COMPOSITIONS THEREOF

(71) Applicants: PTM Therapeutics, Inc., San Mateo, CA (US); The Regents of the University of Michigan, Ann Arbor, MI (US)

(72) Inventors: Tony W. LIANG, San Mateo, CA (US); Miguel Quiros QUESADA, Ann Arbor, MI (US); Jennifer BRAZIL, Ann Arbor, MI (US); Asma NUSRAT, Ann Arbor, MI (US); Charles PARKOS, Ann Arbor, MI (US)

(21) Appl. No.: 19/541,840

(22) Filed: Feb. 17, 2026

Related U.S. Application Data
(63) Continuation of application No. PCT/US2024/043033, filed on Aug. 20, 2024.
(60) Provisional application No. 63/520,703, filed on Aug. 21, 2023.

(51) Int. Cl. A61K 39/395; A61K 31/519; A61K 38/00

(52) U.S. Cl. CPC A61K 39/3955; A61K 31/519; A61K 38/00; C07K 2317/24

(57) ABSTRACT

The present disclosure provides methods of treating disease (e.g., digestive diseases) by administering an antibody or an antigen portion thereof that binds to sLeA and a TNFα inhibitor, as well as pharmaceutical compositions related thereto.

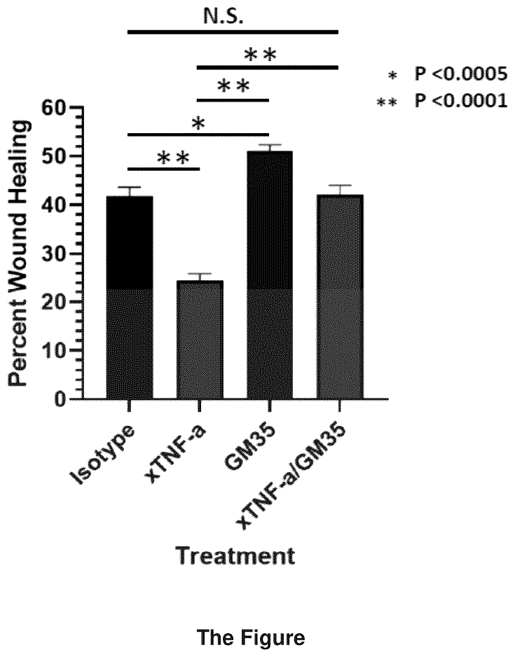

### CROSS-REFERENCE TO RELATED APPLICATIONS

This application is a continuation of International Application No. PCT/US2024/043033, filed Aug. 20, 2024, which claims priority to U.S. Provisional Application No. 63/520,703, filed Aug. 21, 2023, the disclosures of each of which are incorporated herein by reference in their entirety.

### REFERENCE TO AN ELECTRONIC SEQUENCE LISTING

The content of the electronic sequence listing (203462000401 seqlist.xml; Size: 18,659 bytes; and Date of Creation: Feb. 10, 2026) is incorporated herein by reference in its entirety.

### FIELD

The present disclosure relates to methods of, and a pharmaceutical composition for, treating disease (e.g., digestive diseases) using an antibody that binds to sialyl Lewis A and sialyl Lewis C or an antigen-binding fragment thereof and a TNFalpha inhibitor.

### BACKGROUND

Glycans are carbohydrate-based polymers that can be free or attached to proteins (glycoproteins) or lipids (glycolipids). The glycans of glycoproteins are known to be involved in immunity and inflammation, including sialylated glycans on glycoproteins. High expression of sialyl Lewis A (sLeA) have been described in human pancreatic, colon and gastric cell lines as well as in adenocarcinomas of the colon, stomach and pancreas. Additionally, studies have shown sLeA upregulation in concert with CD44v6 in inflamed regions of human colon from patients with ulcerative colitis. Additionally, studies have shown a functional role for sLeA in mucosal inflammation which suggests that modulating sLeA may impact diseases including inflammatory bowel disease (e.g., Crohn's Disease or ulcerative colitis).

Aberrant glycosylation has also been described as one of the hallmarks of cancer and active immune responses. Similar to what has been observed for inflammatory bowel disease tissues, aberrant glycosylation of proteins leads to overexpression of tumor-associated carbohydrate antigens during malignant transformation. Studies have shown that tumor-associated carbohydrate antigens have contributed to various aspects of cancer development and progression, including proliferation, invasion, angiogenesis and metastasis (Fuster, Nat Rev Cancer, 5:526-42 (2005) and Dube, Nat Rev Drug Discovery, 4:477-88 (2005)).

One type of glycosylation are sialic acids and they are typically found to be terminating branches of N-glycans, O-glycans and glycosphingolipids. Within sialic acids, a variety of diversity are created through alpha linkages between the nine-carbon backbone and secondary diversity through modifications at these carbon positions. Examples of sialylated glycans include sLeA, sLeC and sialyl Lewis X.

TNFalpha plays an integral role in inflammatory bowel disease and anti-TNFalpha antibodies or other TNFalpha inhibitor are among the first-line therapeutics to be prescribed to patients with Crohn's disease (CD) or Ulcerative Colitis (UC). However, a significant portion of patients receiving anti-TNFalpha antibody therapy do not respond to treatment or experience a loss of response or intolerance to treatment over time. Studies have also shown that TNFalpha promotes mucosal wound healing in colitis and inhibiting TNFalpha with an anti-TNFalpha antibody therapy or other TNFalpha inhibitor may impair mucosal wound healing contributing to the negative clinical outcomes observed in a portion of the patient population (Bradford et al. J Immunol. 2017 199 (5): 1886-1897).

It has been previously demonstrated that an anti-TNFalpha antibody can inhibit wound healing in an in vivo intestinal injury model (Birki et al. Mucosal Immunol. 2019 12:909-918). Additionally, an antibody that binds both sLeA and sLeC can increase wound healing activity in the same model (Kelm, et al, JCI Insight. 2020; 5 (12): e135843). The present application describes methods of using antibodies that bind both sLeA and sLeC to abrogate the inhibitory wound healing effects of an anti-TNFalpha antibody in intestinal mucosal wounds, including sites of mucosal inflammation and wounds in inflammatory bowel disease. The combination of these two types of therapies may potentially allow for the benefits of the anti-inflammatory effects of an anti-TNFalpha therapy while abrogating of the inhibitory effects on wound healing. Such benefits may include the enhancement of clinical benefits of anti-TNFalpha therapies and/or lowering the dosage needed or reducing the time for patients to see signs of clinical efficacy. Additionally, the combination of a TNFalpha inhibitor with an antibody that binds both sLeA and sLeC may allow for more complete healing of wounds in inflammatory bowel disease, which may lead to remission of the disease and/or longer time for the patient to remain in remission.

The methods of the present application may also be useful in other diseases, disorders, or conditions where anti-TNFalpha therapies are being used by a patient and wound healing activity is needed. Examples of such diseases, disorders or conditions include patients who are on anti-TNFalpha therapies who also need surgical intervention or is undergoing a surgical procedure. The use of antibody that binds to sLeA and sLeC while these patients remain on the anti-TNFalpha therapy may potentially offer clinical benefits directed to increased wound healing in the tissues affected by surgery.

The present invention addresses the need for an alternative antibody therapy that targets sLeA and which are prophylactically and/or therapeutically effective in a variety of disorders and diseases involving wound healing.

### SUMMARY OF THE INVENTION

In some aspects, the present invention provides for methods of treating or ameliorating a symptom of a disease, disorder or condition, comprising administering to a subject a therapeutically effective amount of an antibody that binds to sialyl Lewis A (sLeA) or an antigen-binding fragment thereof in combination with a tumor necrosis factor alpha (TNFα) inhibitor.

In some embodiments, the antibody or antigen-binding fragment thereof also binds to sialyl Lewis C (sLeC).

In other aspects, the methods comprise administering to a subject a therapeutically effective amount of multispecific (e.g., bispecific) antibody comprising a first antigen-binding domain that binds to sLeA and a second antigen-binding domain that binds to TNFα. In some embodiments, the second antigen-binding domain inhibits TNFα activity when bound. In some embodiments, the first antigen-binding domain also binds to sLeC.

In some embodiments according to any of the embodiments described herein, the disease, disorder or condition is a digestive disease or disorder. In some embodiments, the digestive disease or disorder is selected from the group consisting of inflammatory bowel disease, irritable bowel syndrome, pancreatic cancer and colon cancer. In some embodiments, the digestive disease or disorder is inflammatory bowel disease. In some embodiments, the inflammatory bowel disease is Crohn's Disease or ulcerative colitis. In some embodiments, the subject is a patient on an anti-TNFα therapy who also is undergoing a surgical procedure. In some embodiments, the antibody, antigen-binding portion thereof, or first antigen-binding domain does not bind to sialyl Lewis X (sLeX). In some embodiments, the TNFα inhibitor is an antibody or a small molecule. In some embodiments, the TNFα inhibitor is an antibody. In some embodiments, the TNFα inhibitor antibody is selected from the group consisting of infliximab, etanercept, adalimumab, certolizumab, and golimumab. In some embodiments, the second antigen-binding domain comprises the heavy chain variable region (or 3 CDR sequences thereof) and light chain variable region (or 3 CDR sequences thereof) from a single antibody selected from the group consisting of infliximab, etanercept, adalimumab, certolizumab, and golimumab.

In some embodiments according to any of the embodiments described herein, the antibody, antigen-binding portion thereof, or first antigen-binding domain comprises a heavy chain variable region and a light chain variable region, wherein the heavy chain variable region comprises a CDR-H1 comprising the sequence of SEQ ID NO:12, a CDR-HR2 comprising the sequence of SEQ ID NO:13, and a CDR-H3 comprising the sequence of SEQ ID NO: 14; and wherein the light chain variable region comprises a CDR-L1 comprising a sequence selected from the group consisting of SEQ ID Nos: 15-17, a CDR-L2 comprising the sequence of SEQ ID NO: 18, and a CDR-L3 comprising the sequence of SEQ ID NO:19. In some embodiments, the antibody, antigen-binding portion thereof, or first antigen-binding domain is a humanized antibody, antigen-binding portion thereof, or antigen-binding domain. In some embodiments, the antibody, antigen-binding portion thereof, or first antigen-binding domain comprises a heavy chain variable region and a light chain variable region, wherein the heavy chain variable region comprises a sequence having a 90-100% sequence identity to SEQ ID NO:1, SEQ ID NO: 2, SEQ ID NO:3, or SEQ ID NO: 4; and wherein the light chain variable region comprises a sequence having a 90-100% sequence identity to SEQ ID NO: 5, SEQ ID NO: 6, SEQ ID NO: 7, SEQ ID NO:8, SEQ ID NO: 9, SEQ ID NO: 10, or SEQ ID NO: 11. In some embodiments, the antibody, antigen-binding portion thereof, or first antigen-binding domain comprises a heavy chain variable region and a light chain variable region, wherein the heavy chain variable region comprises the sequence of SEQ ID NO:3, and wherein the light chain variable region comprises a sequence selected from the group consisting of SEQ ID Nos: 5-8. In some embodiments, the heavy chain variable region comprises the sequence of SEQ ID NO:3, and the light chain variable region comprises the sequence of SEQ ID NO:8. In some embodiments, the antibody, antigen-binding portion thereof, or first antigen-binding domain comprises a heavy chain variable region and a light chain variable region, wherein the heavy chain variable region comprises the sequence of SEQ ID NO:4, and wherein the light chain variable region comprises a sequence selected from the group consisting of SEQ ID Nos: 9-10. In some embodiments, the antibody, antigen-binding portion thereof, or first antigen-binding domain comprises a heavy chain variable region and a light chain variable region, wherein the heavy chain variable region comprises a sequence having a 90-100% sequence identity to SEQ ID NO:3, and wherein the light chain variable region comprises a sequence having a 90-100% sequence identity to a sequence selected from the group consisting of SEQ ID Nos: 5-11. In some embodiments, the subject is a human.

In other aspects, the present invention provides an antibody that binds to sialyl Lewis A (sLeA) or an antigen-binding fragment thereof for use in a method of treating or ameliorating a symptom of a disease, disorder or condition (e.g., a digestive disease or disorder disclosed herein), said method comprising administering a therapeutically effective amount of the antibody in combination with a tumor necrosis factor alpha (TNFα) inhibitor (e.g., according to any one of the methods disclosed herein).

In other aspects, the present invention provides the use of an antibody that binds to sialyl Lewis A (sLeA) or an antigen-binding fragment thereof in the manufacture of a medicament for treating or ameliorating a symptom of a disease, disorder or condition (e.g., a digestive disease or disorder disclosed herein), wherein a therapeutically effective amount of the medicament is to be administered in combination with a tumor necrosis factor alpha (TNFα) inhibitor. In other aspects, the present invention provides use of an antibody that binds to sialyl Lewis A (sLeA) or an antigen-binding fragment thereof in the manufacture of a medicament for treating or ameliorating a symptom of a disease, disorder or condition (e.g., a digestive disease or disorder disclosed herein), wherein the medicament further comprises a tumor necrosis factor alpha (TNFα) inhibitor.

### BRIEF DESCRIPTION OF THE DRAWING

The FIGURE illustrates in vivo wound healing activity in a model of mechanical wounding in mice. Wounds were treated with either 250 μg of isotype control antibody on the day of injury (Day 0) and one day after injury (Day 1); 500 μg of anti-murine TNFalpha antibody on the day before injury (Day-1), Day 0, and Day 1; 250 μg of GM35 on Day 0 and Day 1; or 500 μg of anti-murine TNFalpha antibody on Day-1, 500 μg anti-murine TNFalpha antibody and 250 μg GM35 on Day 0 and Day 1. Wound healing activity is calculated by area measurement of the wounds on Day 1 and Day 3 and the following formula: 100−((area on Day 3/area on Day1)*100). Data are shown as mean±SEM and were analyzed by 1-way ANOVA followed by Tukey's post hoc testing.

### DETAILED DESCRIPTION OF THE INVENTION

As used herein, the articles “a” and “an” refer to one or more than one (i.e., at least one) of the grammatical object of the article. By way of example, “an element” means one element or more than one element.

The term “antibody” is intended to encompass antibodies, fragments, specified portions and variants thereof, including single chain antibodies and fragments thereof, derived from an antibody of the present invention. Antibodies include antibody fragments, antibody variants, monoclonal antibodies, polyclonal antibodies, and recombinant antibodies. Antibodies can be generated in mice, rats, rabbits, or humans.

The antibodies can be full-length or can comprise a fragment (or fragments) of the antibody having an antigen portion, including, but not limited to, Fab, Fab′, and F(ab′)₂, facb, pFc¹, Fd, dAb fragment, an isolated CDR, diabodies, triabodies, tetrabodies, linear antibodies, single-chain antibody molecules, bispecific and multi-specific antibodies formed from antibody fragments.

In some embodiments, the antibodies comprise all or a portion of a constant region of an antibody. The constant region is an isotype selected from IgA (e.g., IgAQ1 or IgA2), IgD, IgE, IgG (e.g., IgG1, IgG2, IgG3, or IgG4), IgM. As used herein, the “constant region” of an antibody includes the natural constant region, allotypes or natural variants, such as D356E and L358M or A431G in human IgG1. See, e.g., Jefferies and Lefranc, MAbs, 1 (4): 332-338 (July-August 2009).

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies of the present invention can be prepared using a wide variety of techniques known in the art including the use of hybridoma, recombinant and phage display technologies, or a combination thereof.

The term “chimeric” antibody as used herein refers to an antibody having variable sequences derived from a non-human immunoglobulin, such as a rat or a mouse antibody, and a human immunoglobulin constant region, typically chosen from a human immunoglobulin template. Methods for producing chimeric antibodies are known in the art.

“Humanized” form of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody can also comprise at least a portion of an immunoglobulin constant region (Fc), typically that or a human immunoglobulin consensus sequence. Methods of antibody humanization are known in the art.

An “effective amount” as used herein, refers to a dose of the antibody or pharmaceutical composition that is sufficient to reduce the symptoms and signs of diseases of the digestive system, including inflammatory bowel disease or cancers of the digestive system. Such symptoms of inflammatory bowel disease include diarrhea, weight loss, bloody diarrhea, bloody stool, pain, anemia, fatigue, rectal bleeding, and abdominal cramps. Symptoms of cancers of the digestive system include weight loss, pain and detectable mass, either clinically as a palpable mass or radiologically or through other imaging techniques. The term “effective amount” and “therapeutically effective amount” are used interchangeably. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic or other effective agents, and a single agent may be considered to be given in an effective amount if, in conjunction with one or more other agents, a desirable result may be or is achieved. While individual needs vary, determination of optimal ranges of effective amounts of each component is within the skill of the art. Typical dosages comprise 0.1 to 100 mg/kg/body weight. The preferred dosages comprise 1 to 100 mg/kg/body weight. The most preferred dosages comprise 10 to 100 mg/kg/body weight.

The term “subject” or “individual” can refer to a vertebrate having a disease of the digestive system, such as a vertebrate having inflammatory bowel disease or cancer of the digestive system. Subjects include all warm-blooded animals, such as mammals, such as a rodent, preferably a primate or non-human primate, and more preferably, a human. The term subject includes domesticated animals, such as cats, dogs, etc., livestock (for example, cattle, horses, pigs, sheep, goats, etc.), and laboratory animals (for example, mouse, rabbit, rat, gerbil, guinea pig, etc.). Thus, veterinary uses and medical or pharmaceutical formulations are contemplated herein.

One of skill in the art will recognize that antibodies are “modular” in nature. Throughout the disclosure, various specific embodiments of the various “modules” composing the antibodies of the present invention are described. As specific non-limiting examples, various embodiments of variable heavy chain CDRs, variable heavy chains, variable light chain CDRs, and variable light chains are described. It is intended that all of the specific embodiments may be combined with each other as though each specific combination were explicitly described individually.

Humanized antibodies of the present invention can comprise a heavy chain variable region from Table 1 below and additionally, can comprise a light chain variable region from Table 2 below. Such variable regions can be incorporated into a human IgG1 backbone using methods that are well known in the art.

Table 1. Heavy Chain Variable Region Sequences

| | | |
|---|---|---|
| HvCh1 | SEQ ID NO: 1 | EVQLVESGGGLVQPGGSLRLSCAASGFTFSTNAMS |
| | | WVRQAPGKGLEWVSRLRPKSDNYATYYADSVKG |
| | | RFTISRDNSKNTLYLQMNSLRAEDTAVYYCAKGT |
| | | GFWGQGTTVTVSS |
| HvCh2 | SEQ ID NO: 2 | EVQLVESGGGLVQPGGSLRLSCAASGFTFSTNAMS |
| | | WVRQAPGKGLEWVARLRPKSDNYATYYADSVKG |
| | | RFTISRDNSKNTLYLQMNSLRAEDTAVYYCVTGT |
| | | GFWGQGTTLTVSS |
| HvCh3 | SEQ ID NO: 3 | EVQLVESGGGLVQPGGSLRLSCAASGFTFSTNAMSWVR |
| | | QAPGKGLEWVARLRPKSDNYATYYADSVKGRFTISRD |
| | | DSKNTLYLQMNSLRAEDTAVYYCVTGTGFWGQGTTLT |
| | | VSS |
| HvCh4 | SEQ ID NO: 4 | EVQLVESGGGLVQPGGSLRLSCAASGFTFSTNAMS |
| | | WVRQAPGKGLEWVARLRPKSDNYATYYADSVKG |
| | | RFTISRDDSTSTLYLQMNSLRAEDTAVYYCVTGTG |
| | | FWGQGTTLTVSS |

Table 2. Light Chain Variable Region Sequences

| | | |
|---|---|---|
| LiCh1 | SEQ ID NO: 5 | DIVMTQSPDSLAVSLGERATINCKSSQSLLNSGNQ |
| | | KNYLTWYQQKPGQPPKLLIYWTSTRESGVPDRFS |
| | | GSGSGTDFTLTISSLQAEDVAVYYCQNDYTSPYTF |
| | | GQGTKLEIK |
| LiCh2 | SEQ ID NO: 6 | DIVMTQSPDSLAVSLGERVTMNCKSSQSLLNSGNQ |
| | | KNYLTWYQQKPGQPPKLLIYWTSTRESGVPDRFS |
| | | GSGSGTDFTLTISSVQAEDVAVYYCQNDYTSPYTF |
| | | GQGTKLEIK |
| LiCh3 | SEQ ID NO: 7 | DIVMTQSPDSLAVSLGERATINCKSSQSLLNSGNQ |
| | | KNYLTWYQQKPGQPPKLLFYWTSTRESGVPDRFS |
| | | GSGSGTDFTLTISSLQAEDVAVYYCQNDYTSPYTF |
| | | GQGTKLEIK |
| LiCh4 | SEQ ID NO: 8 | DIVMTQSPDSLAVSLGERVTMNCKSSQSLLNSGNQ |
| | | KNYLTWYQQKPGQPPKLLFYWTSTRESGVPDRFS |
| | | GSGSGTDFTLTISSVQAEDVAVYYCQNDYTSPYTF |
| | | GQGTKLEIK |
| LiCh5 | SEQ ID NO: 9 | DIVMTQSPDSLAVSLGERVTMNCKSSQSLLNSGNQ |
| | | KNYLTWYQQKPGQPPKLLFYWTSTRESGVPDRFS |
| | | GSGSGTDFTLTISSVQAEDLAVYYCQNDYTSPYTF |
| | | GQGTKLEIK |
| LiCh6 | SEQ ID NO: 10 | DIVMTQSPDSLAVSLGERVTMNCKSSQSLLQSGNQ |
| | | KNYLTWYQQKPGQPPKLLFYWTSTRESGVPDRFS |
| | | GSGSGTDFTLTISSVQAEDLAVYYCQNDYTSPYTF |
| | | GQGTKLEIK |
| LiCh7 | SEQ ID NO: 11 | DIVMTQSPDSLAVSLGERVTMNCKSSQSLLSSGNQ |
| | | KNYLTWYQQKPGQPPKLLFYWTSTRESGVPDRFS |
| | | GSGSGTDFTLTISSVQAEDLAVYYCQNDYTSPYTF |
| | | GQGTKLEIK |

In other embodiments, the humanized antibodies of the present invention can comprise heavy and light chain CDR sequences selected from Tables 3 and 4 below:

Table 3. Heavy Chain CDR sequences

| | | |
|---|---|---|
| H-CDR1 | SEQ ID NO: 12 | GFTFSTNAMS |
| H-CDR2 | SEQ ID NO: 13 | RLRPKSDNYATY |
| H-CDR3 | SEQ ID NO: 14 | VTGTGF |

Table 4. Light Chain CDR sequences

| | | |
|---|---|---|
| L-CDR1 | SEQ ID NO: 15 | KSSQSLLNSGNQKNYLT |
| L-CDR1B | SEQ ID NO: 16 | KSSQSLLQSGNQKNYLT |
| L-CDR1C | SEQ ID NO: 17 | KSSQSLLSSGNQKNYLT |
| L-CDR2 | SEQ ID NO: 18 | WTSTRES |
| L-CDR3 | SEQ ID NO: 19 | QNDYTSPYT |

The present invention includes antibodies and fragments that specifically binds to sLeA and sLeC, but not sialyl Lewis X (sLeX), compositions comprising antibodies, polynucleotides encoding anti-sLeA/sLeC, but not sLeX antibodies, polynucleotides encoding such antibodies, methods and compositions useful for making such antibodies and binding fragments, and various methods using the same. Glycan structures of sLeA, sLeC, and sLeX are shown in Table 5 below.

TABLE 5
Glycan structure of sialyl Lewis A,
sialyl Lewis C, and sialyl Lewis X

| Glycan name | Glycan structure |
|---|---|
| sialyl Lewis A (sLeA) | NeuAc α2,3 Gal β1,3 [Fucα1,4] GlcNAc |
| sialyl Lewis C (sLeC) | NeuAc α2,3 Gal β1,3 GlcNAc |
| sialyl Lewis X (sLeX) | NeuAc α2,3 Galβ1,4 [Fucα1,3] GlcNAc |

Antibodies against carbohydrate/glycans can be unsuitable for pharmaceutical development because of its generally lower binding affinity. Methods for determining binding affinities for antibodies are known in the art. Generally, the binding affinity to a particular target or substrate is determined by the relationship between the on rate (kₐ (M⁻¹s⁻¹)) and off rate (k_{d} (s⁻¹)) to produce an equilibrium dissociation constant (K_{D}). The lower the K_{D}, the higher the affinity. Examples of methods for determining antibody affinity include assays utilizing the Octet system (Fortébio) or the Biacore system (GE Healthcare) or other systems instrumentation that determine association and dissociation constants. Accordingly, the present invention comprises antibodies having binding affinity to sLeA of a K_{D} of about 500 μM or less and binding affinity to sLeC of a K_{D} of about 500 μM or less, and no binding to sLeX. In non-limiting examples, antibodies of the present invention comprise a binding affinity to sLeA of a K_{D} of 100 μM or less, 90 μM or less, 80 UM or less, 70 μM or less, 60 μM or less, 50 UM or less, 40 μM or less, 30 μM or less, 20 μM or less, 10 UM or less, 1 μM or less, or 0.1 μM or less, and a binding affinity to sLeC of a K_{D} of 100 μM or less, 90 μM or less, 80 μM or less, 70 μM or less, 60 μM or less, 50 μM or less, 40 μM or less, 30 μM or less, 20 μM or less, 10 μM or less, 1 μM or less, or 0.1 μM or less, and no binding to sLeX. In one embodiment, antibodies that are useful in the present invention have a binding affinity to their glycan targets of a K_{D} of 41 μM or less for sLeA and a K_{D} of 70 μM or less for sLeC and no binding to sialyl Lewis X (sLeX). In some embodiments, an antibody of the present disclosure does not bind to a particular target (e.g., sLeX) if the binding affinity of the antibody to the target is of a K_{D} of about 1 mM or higher.

Antibodies of the present invention may also be used in methods of aiding diagnosis of disease, such as diagnosis of cancer in an individual. Such cancers include cancers of the digestive system such as pancreatic or colon cancer. Such methods include using the antibodies of the present invention to determine the level of sLeA and/or sLeC binding in an individual or specific tissue in an individual. As used herein, methods for “aiding diagnosis” means that these methods assist in making a clinical determination regarding the classification, or nature, or cancer, and may or may not be conclusive with respect to the definitive diagnosis. Accordingly, a method of aiding diagnosis of cancer can comprise the step of detecting the level of sLeA and/or sLeC in a biological sample from the individual and/or determining the level of sLeA and/or sLeC in the sample. Antibodies recognizing the epitope or a portion thereof may also be used to create diagnostic immunoassays for detecting antigenic determinant released in the bodily fluids, including but not limited to blood, saliva, urine, pulmonary fluid, or ascites fluid. Similarly, such immunoassays using the antibodies of the present invention may be used to monitor the efficacy of treatment and/or disease remission. In such instances, the presence and/or levels of the antigenic determinant reactive to the antibody of the present invention may be useful as a biomarker in order to monitor disease activity and/or progression.

In some embodiments, an antibody of the present disclosure may also be used in a method of detecting the presence and/or measuring the level of sLeA and/or sLeC in an individual or specific tissue in an individual.

Antibodies of the present invention may be used for therapeutic purposes in individuals with diseases or disorders with delayed wound healing. In some embodiments, non-limiting examples of tissues that may be in need of increased wound healing activity with the use of pharmaceutical compositions are liver tissues, kidney tissues, lung tissues, heart tissues, skin tissues, and eye tissues, including corneal tissue.

In one embodiment, a method for increasing wound healing activity is provided, which comprises administering an anti-sLeA antibody or antigen binding fragment there of as part of a pharmaceutical formulation.

In some embodiments, the antibodies binding to sLeA of the present invention may be used alone for the treatment or ameliorating one or more disease symptom. In other embodiments, the antibodies of the present invention may be used in combination with other therapeutics or pharmaceutical agents to treat or ameliorate one or more disease symptom.

In some embodiments, an antibody of the present disclosure may also be used in a method of detecting the presence and/or measuring the level of sLeA and/or sLeC in an individual or specific tissue in an individual.

Certain aspects of the present disclosure relate to polynucleotides (e.g., isolated polynucleotides) and/or vectors (e.g., expression vectors) encoding an antibody of the present disclosure, as well as host cells (e.g., isolated host cells) comprising the polynucleotides or vectors. In some embodiments, the host cell is a prokaryotic host cell, such as a bacterial host cell (e.g., E. coli). Suitable prokaryotic host cells include without limitation eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, etc. In some embodiments, the host cell is a eukaryotic host cell such as yeast, fungi, insect, plant, animal, human, or nucleated cells from other multicellular organisms. For example, filamentous fungi or yeast are suitable cloning or expression hosts for antibody-encoding vectors. Saccharomyces cerevisiae, or common baker's yeast, is the most commonly used among lower eukaryotic host microorganisms. Suitable host cells for the expression of glycosylated antibody are also derived from multicellular organisms (invertebrates and vertebrates). Examples of invertebrate cells include plant and insect cells. Examples of vertebrate or mammalian cells include, e.g., monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; a human hepatoma line (Hep G2); Chinese hamster ovary (CHO) cells, including DHFR⁻ CHO cells (Urlaub et al., Proc. Natl. Acad. Sci. USA 77:4216 (1980)); and myeloma cell lines such as NS0 and Sp2/0.

Other aspects of the present disclosure relate to methods of production using a host cell of the present disclosure. Antibodies or antigen-binding portions thereof can be produced using recombinant methods. For recombinant production of an antibody or antigen-binding portion, nucleic acid encoding the antibody/portion is isolated and inserted into a vector for further cloning (amplification of the DNA) or for expression. DNA encoding the antibody can be isolated and sequenced using conventional procedures (e.g., by using oligonucleotide probes that are capable of binding specifically to genes encoding the heavy and light chains of the antibody). The vector components generally include, but are not limited to, one or more of the following: a signal sequence, an origin of replication, one or more marker genes, an enhancer element, a promoter, and a transcription termination sequence.

Other aspects of the present disclosure relate to methods of administering an anti-sLeA antibody that may also bind to sLeA or antigen binding fragment thereof with a tumor necrosis factor alpha (TNFα) inhibitor as part of a pharmaceutical formulation. TNFα inhibitors are known in the art and have been approved by the FDA to treat various diseases and disorders including diseases and disorders of the digestive system, such as Crohn's Disease or Ulcerative Colitis. Non-limiting examples of TNFα inhibitors include antibody therapeutics such as infliximab (REMICADE®; Janssen), adalimumab (HUMIRA®; Abbvie), etanercept (ENBREL®; Amgen), golimumab (SIMPONI®; Janssen), and certolizumab (CIMZIA®; UCB). Other modalities of TNFα inhibitors may be used in the methods of the present invention including anti-TNFα antigen binding fragments, small molecule inhibitors of TNFα, and other inhibitors of TNFα.

Another aspect of the present invention relates to the use of a bispecific antibody or antigen binding fragment thereof, where one antigen binding fragment binds to sLeA and the other antigen binding fragment binds to TNFα. In some embodiments for the bispecific antibody of the present invention, the antigen binding fragment that binds to TNFα inhibits TNFα activity when bound. In other embodiments, the antigen binding fragment that binds to sLeA also binds to sLeC.

Various formulations of antibodies of the present invention or fragments thereof may be used for administration. In some embodiments, the antibodies of the present invention or fragments thereof may be administered neat. In addition to the pharmacologically active agent, the compositions of the present invention may contain suitable pharmaceutically acceptable carriers comprising excipients and auxiliaries that are well known in the art and are relatively inert substances that facilitate administration of a pharmacologically effective substance or which facilitate processing of the active compounds into preparations that can be used pharmaceutically for delivery to the site of action. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers.

Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington, The Science and Practice of Pharmacy 20th Ed. Mack Publishing (2000).

Suitable formulations for oral administration include hard or soft gelatin capsules, pills, tablets, including coated tablets, elixirs, suspensions, syrups or inhalations and controlled release forms thereof. Additionally, oral administration of a peptide or protein therapeutic such as an antibody therapeutic can be coated with an active ingredient or formulated to be resistant against digestion in the stomach. Oral administration may also include sublingual and buccal administration.

Generally, pharmaceutical agents may be formulated for administration by injection (e.g., intraperitoneally, intravenously, subcutaneously, intramuscularly, etc.), although other forms of parenteral administration (e.g., local administration, topical, intranasal, intrapulmonary, ocular, and rectal) can be also used. Accordingly, antibodies of the present invention are preferably combined with pharmaceutically acceptable vehicles such as saline, Ringer's solution, dextrose solution, and the like. The pharmaceutical composition may be administered using any device that may help migration or deposition of an active component to a target cell.

The particular dosage regimen, i.e., dose, timing and repetition, will depend on the particular individual and that individual's medical history and mode of administration. One of ordinary skill in the art may determine a dose/dosing regimen that is suitable. Generally, a dose of at least about 0.1 mg/kg body weight, or more preferably, at least about 1 mg/kg body weight, or at least about 5 mg/kg body weight, even more preferably at least about 10 mg/kg body weight or at least about 20 mg/kg body weight is administered by injection.

In some embodiments, one or more doses of the antibodies of the present invention or fragments thereof will be administered during a course of treatment. Empirical considerations, such as half-life, generally will contribute to the determination of the dosage. Antibodies, which are compatible with the human immune system, such as humanized antibodies or fully human antibodies ma y be used to prolong half-life of the antibody and prevent the antibody being attacked by the host's immune system. Frequency of administration may be determined and adjusted over the course of therapy and may be based on the reduction of one of more clinical symptoms of the disease. Alternatively, sustained continuous release formulations of the antibodies of the present invention may be appropriate. Various formulations and devices for achieving sustained release are known in the art.

In some aspects, the methods herein comprising administration of a therapeutically effective amount of an antibody that binds to sLeA (and optionally sLeC) in combination with a TNFalpha inhibitor. Such combination therapies noted herein encompass combined administration (where two or more therapeutic agents are included in the same or separate formulations), and separate administration, in which case, administration of the antibody of the invention can occur prior to, simultaneously, and/or following, administration of the TNFalpha inhibitor.

The following examples are provided to illustrate, but not to limit the invention.

### EXAMPLES

### Example 1. Generation of Humanized Antibody Clones

Humanized antibodies, with a heavy chain variable region comprising one of the heavy chain variable regions from Table 1 above and a light chain variable region comprising one of the light chain variable regions from Table 2 above were synthesized and incorporated into an IgG1 backbone. Heavy and light chain variable region pairs that were incorporated into an IgG1 backbone and synthesized were the ones in the Table 6 below.

TABLE 6
Humanized Antibody Clones

| Humanized | | | | |
|---|---|---|---|---|
| Antibody | Heavy Chain | SEQ | Light Chain | SEQ |
| Clone | Variable Region | ID NO: | Variable Region | ID NO: |
| 4764 | HvCh1 | 1 | LiCh1 | 5 |
| 4765 | HvCh2 | 2 | LiCh1 | 5 |
| 4766 | HvCh2 | 2 | LiCh2 | 6 |
| 4767 | HvCh2 | 2 | LiCh3 | 7 |
| 4768 | HvCh2 | 2 | LiCh4 | 8 |
| 4769 | HvCh3 | 3 | LiCh1 | 5 |
| 4770 | HvCh3 | 3 | LiCh2 | 6 |
| 4771 | HvCh3 | 3 | LiCh3 | 7 |
| 4772 | HvCh3 | 3 | LiCh4 | 8 |

Humanized antibodies with the above combinations of heavy and light chain variable region pairs were produced by transfecting the plasmids into mammalian cells and purified using affinity chromatography.

Of the nine humanized antibody clones in Table 6, two antibody clones (4764 and 4767) showed weak expression, resulting in less than 1 mg of antibody after purification. The production levels for these two clones were deemed too low for practical production cell line optimization. The remaining seven clones were then screened for binding affinity to sLeA, sLeC and sLeX.

### Example 2. Binding Affinity to Sialyl Lewis A (sLeA) and Sialyl Lewis C (sLeC)

An in vitro evaluation of the binding affinity to sLeA and sLeC of the humanized antibodies from Example 1 were performed using a Biocore T100 SPR biosensor using a CM7 sensor chip with PBS-p (0.005% Tween-20) running buffer at 25 degrees Celsius. The antibodies were couples to ˜30,000 RU at 50 ug/ml in 10 mM NaAcetate pH 5.0 over an NHS/EDC activated surface. A reference surface was activated and blocked to serve as a control. Carbohydrate samples (sLeA, sLeC and sialyl Lewis X (all from Dextra Labs)) were dissolved in PBS-p running buffer up to a stock concentration of 10 mM. Each was then prepared in a 2-fold dilution series up to 300 μM and each concentration series was tested over the antibody surfaces. Response data were processed by subtracting the responses from the reference surfaces as well as a buffer injection. Binding constants were determined at 25 degrees Celsius.

We tested a murine antibody (GM35) described in Brazil, J Immunol 191:4804-4817 (2013) for binding affinities for sLeA, sLeC and sLeX using the methods described above. The results for this antibody did not show binding to sLeX, had a K_{D} for sLeA of 41.5 μM to 46.8 μM, and a K_{D} for sLeC of 68.8 μM to 78.2 μM. Comparatively, another antibody that was tested was NS19-9 (Dako, Carpenteria, CA), an antibody known to bind to sLeA, which did not show binding to sLeX, had a K_{D} for sLeA of 39.5 μM, and a K_{D} for sLeC of 1.5 mM.

### Example 3. Binding Affinity to Sialyl Lewis A (sLeA) and Sialyl Lewis C (sLeC) for Humanized Antibody Clones

Seven of the humanized antibody clones from Example 1 were tested for binding affinity to sLeA, sLeC and sLeA. Methods used were similar to those described in Example 2 above, except with sLeX only at one concentration of 300 μM. Mean binding K_{D} for sLeA and sLeC for each of the seven humanized antibody clones are shown in Table 7 below.

TABLE 7
Binding Affinity for Humanized Antibody Clones

| Humanized | sLeA Mean K_{D} | sLeC Mean K_{D} |
|---|---|---|
| Antibody Clone | in μM | in μM |
| 4765 | 731.7 | 306.3 |
| 4766 | 620.7 | 538.0 |
| 4768 | 552.0 | 460.0 |
| 4769 | 81.3 | 103.7 |
| 4770 | 69.1 | 93.2 |
| 4771 | 59.2 | 85.4 |
| 4772 | 48.3 | 77.1 |
| GM35 | 44.5 | 73.4 |

None of the above antibody clones showed binding to sLeX in three independent runs at a concentration of 300 μM of the carbohydrate. From the above binding affinity data, humanized antibody clones with HvCh3 variable regions had the highest affinity for both sLeA and sLeC.

We also characterized the binding affinity (using the same methods as those described for the humanized antibody clones) of GM35 to sLeA and sLeC. The results of the binding affinities are also summarized in Table 7 above. Similar to the humanized antibody clones, GM35 also did not show binding to sLeX.

### Example 4. In Vitro Wound Healing Activity

It has been previously demonstrated that an anti-TNFalpha antibody can inhibit wound healing during an in vivo intestinal injury model (Birki et al. Mucosal Immunol. 2019 12:909-918). Additionally, an antibody that binds both sLeA and sLeC can increase wound healing activity in the same model (Kelm, et al, JCI Insight. 2020; 5 (12): e135843). Such antibodies include the ones described in the present application. Here we tested whether the combination of an anti-TNFalpha antibody and an antibody that binds both sLeA and sLeC can abrogate the inhibition of wound healing activity seen with an anti-TNFalpha antibody alone during an in vivo intestinal injury model.

Mice were anesthetized by an intraperitoneal injection of a ketamine (100 mg/kg)/xylazine (10 mg/kg) solution. Biopsy-induced injuries of the colonic mucosa were made along the mesenteric artery using a high-resolution, miniaturized colonoscope system equipped with a biopsy forceps (Colorview Veterinary Endoscope, Karl Storz). For each animal, 5-6 lesions were generated using this system. Antibodies were injected intraperitoneally as follows: (a) 250 μg of isotype control antibody (BD Biosciences, Catalog #554721) on the day of injury (Day 0) and one day after injury (Day 1); (b) 500 μg of anti-murine TNFalpha antibody (InVivoMAb, Catalog #BE0058) on the day before injury (Day-1), Day 0, and Day 1; (c) 250 μg of GM35 on Day 0 and Day 1; and (d) 500 μg of anti-murine TNFalpha antibody on Day-1, 500 μg anti-murine TNFalpha antibody and 250 μg GM35 on Day 0 and Day 1. Endoscopic procedures were viewed on Day 1 and 3 after injury with high-resolution images (1024×768 pixels) on a flat panel monitor. Still images corresponding to specific wounds were taken from the endoscopic videos and area of injury was compared on Day 3 vs. Day 1 after wounding. Wound healing results were then calculated as percentage of wound repair; formula=100−((area on Day 3/area on Day 1)*100). Data are shown as mean±SEM and were analyzed by 1-way ANOVA followed by Tukey's post hoc testing.

As seen in The FIGURE, in conditions treated with an anti-murine TNFalpha only, there was a significant decrease in wound healing activity compared to the isotype control. In conditions treated with GM35, significant increase in wound healing activity compared to the isotype control was observed. Additionally, significant increase in wound healing activity was also observed comparing the anti-TNFalpha and GM35 treatment groups. Interestingly, in the combination of an anti-murine TNFalpha and GM35 treatment groups, the inhibition of wound healing activity was abrogated to a level of wound healing activity that was comparable to isotype control. This data suggests that the addition of an antibody that binds to sLeA and sLeC can overcome the inhibition of wound healing activity of an anti-TNFalpha antibody in the context of intestinal mucosal injury.

SEQUENCES

SEQ ID NO: 1; Amino acid sequence of HvCh1
EVQLVESGGGLVQPGGSLRLSCAASGFTFSTNAMSWVRQAPGKGLEWVSRLRPKSDNY
ATYYADSVKGRFTISRDNSKNTLYLQMNSLRAEDTAVYYCAKGTGFWGQGTTVTVSS
SEQ ID NO: 2; Amino acid sequence of HvCh2
EVQLVESGGGLVQPGGSLRLSCAASGFTFSTNAMSWVRQAPGKGLEWVARLRPKSDN
YATYYADSVKGRFTISRDNSKNTLYLQMNSLRAEDTAVYYCVTGTGFWGQGTTLTVSS
SEQ ID NO: 3; Amino acid sequence of HvCh3
EVQLVESGGGLVQPGGSLRLSCAASGFTFSTNAMSWVRQAPGKGLEWVARLRPKSDN
YATYYADSVKGRFTISRDDSKNTLYLQMNSLRAEDTAVYYCVTGTGFWGQGTTLTVSS
SEQ ID NO: 4; Amino acid sequence of HvCh4
EVQLVESGGGLVQPGGSLRLSCAASGFTFSTNAMSWVRQAPGKGLEWVARLRPK
SDNYATYYADSVKGRFTISRDDSTSTLYLQMNSLRAEDTAVYYCVTGTGFWGQG
TTLTVSS
SEQ ID NO: 5; Amino acid sequence of LiCh1
DIVMTQSPDSLAVSLGERATINCKSSQSLLNSGNQKNYLTWYQQKPGQPPKLLIYWTST
RESGVPDRFSGSGSGTDFTLTISSLQAEDVAVYYCQNDYTSPYTFGQGTKLEIK
SEQ ID NO: 6; Amino acid sequence of LiCh2
DIVMTQSPDSLAVSLGERVTMNCKSSQSLLNSGNQKNYLTWYQQKPGQPPKLLIYWTS
TRESGVPDRFSGSGSGTDFTLTISSVQAEDVAVYYCQNDYTSPYTFGQGTKLEIK
SEQ ID NO: 7; Amino acid sequence of LiCh3
DIVMTQSPDSLAVSLGERATINCKSSQSLLNSGNQKNYLTWYQQKPGQPPKLLFYWTST
RESGVPDRFSGSGSGTDFTLTISSLQAEDVAVYYCQNDYTSPYTFGQGTKLEIK
SEQ ID NO: 8; Amino acid sequence of LiCh4
DIVMTQSPDSLAVSLGERVTMNCKSSQSLLNSGNQKNYLTWYQQKPGQPPKLLFYWTS
TRESGVPDRFSGSGSGTDFTLTISSVQAEDVAVYYCQNDYTSPYTFGQGTKLEIK
SEQ ID NO: 9; Amino acid sequence of LiCh5
DIVMTQSPDSLAVSLGERVTMNCKSSQSLLNSGNQKNYLTWYQQKPGQPPKLLFYWTS
TRESGVPDRFSGSGSGTDFTLTISSVQAEDLA VYYCQNDYTSPYTFGQGTKLEIK
SEQ ID NO: 10; Amino acid sequence of LiCh6
DIVMTQSPDSLAVSLGERVTMNCKSSQSLLQSGNQKNYLTWYQQKPGQPPKLLFYWTS
TRESGVPDRFSGSGSGTDFTLTISSVQAEDLAVYYCQNDYTSPYTFGQGTKLEIK
SEQ ID NO: 11; Amino acid sequence of LiCh7
DIVMTQSPDSLAVSLGERVTMNCKSSQSLLSSGNQKNYLTWYQQKPGQPPKLLFYWTS
TRESGVPDRFSGSGSGTDFTLTISSVQAEDLAVYYCQNDYTSPYTFGQGTKLEIK
SEQ ID NO: 12; Amino acid sequence of heavy chain CDR-1
GFTFSTNAMS
SEQ ID NO: 13; Amino acid sequence of heavy chain CDR-2
RLRPKSDNYATY
SEQ ID NO: 14; Amino acid sequence of heavy chain CDR-3
VTGTGF
SEQ ID NO: 15; Amino acid sequence of light chain CDR-1
KSSQSLLNSGNQKNYLT
SEQ ID NO: 16; Amino acid sequence of light chain CDR-1B
KSSQSLLQSGNQKNYLT
SEQ ID NO: 17; Amino acid sequence of light chain CDR-1C
KSSQSLLSSGNQKNYLT
SEQ ID NO: 18; Amino acid sequence of light chain CDR-2
WTSTRES
SEQ ID NO: 19; Amino acid sequence of light chain CDR-3
QNDYTSPYT

1. A method for treating or ameliorating a symptom of a disease, disorder or condition comprising administering to a subject a therapeutically effective amount of an antibody that binds to sialyl Lewis A (sLeA) or an antigen-binding fragment thereof in combination with a tumor necrosis factor alpha (TNFα) inhibitor.

2. The method of claim 1, wherein the antibody or antigen-binding fragment thereof also binds to sialyl Lewis C (sLeC).

3. The method of claim 1, wherein the disease, disorder or condition is a digestive disease or disorder.

4. The method of claim 3, wherein the digestive disease or disorder is selected from the group consisting of inflammatory bowel disease, irritable bowel syndrome, pancreatic cancer and colon cancer.

5. The method of claim 4, wherein the digestive disease or disorder is inflammatory bowel disease.

6. The method of claim 5, wherein the inflammatory bowel disease is Crohn's Disease or ulcerative colitis.

7. The method of claim 1, wherein the subject is a patient on an anti-TNFα therapy who also is undergoing a surgical procedure.

8. The method of claim 1, wherein the antibody or antigen-binding portion thereof does not bind to sialyl Lewis X (sLeX).

9. The method of claim 1, wherein the TNFα inhibitor is an antibody or a small molecule.

10. The method of claim 9, wherein the TNFα inhibitor is an antibody.

11. The method of claim 10, wherein the TNFα inhibitor antibody is selected from the group consisting of infliximab, etanercept, adalimumab, certolizumab, and golimumab.

12. The method of claim 1, wherein the antibody or antigen-binding portion thereof comprises a heavy chain variable region and a light chain variable region, wherein the heavy chain variable region comprises a CDR-H1 comprising the sequence of SEQ ID NO: 12, a CDR-HR2 comprising the sequence of SEQ ID NO: 13, and a CDR-H3 comprising the sequence of SEQ ID NO: 14; and wherein the light chain variable region comprises a CDR-L1 comprising a sequence selected from the group consisting of SEQ ID Nos: 15-17, a CDR-L2 comprising the sequence of SEQ ID NO: 18, and a CDR-L3 comprising the sequence of SEQ ID NO:19.

13. The method of claim 1, wherein the antibody or antigen-binding fragment thereof is a humanized antibody or humanized antigen-binding fragment.

14. The method of claim 1, wherein the antibody or antigen binding fragment comprises a heavy chain variable region and a light chain variable region, wherein the heavy chain variable region comprises a sequence having a 90-100% sequence identity to SEQ ID NO:1, SEQ ID NO: 2, SEQ ID NO:3, or SEQ ID NO: 4; and wherein the light chain variable region comprises a sequence having a 90-100% sequence identity to SEQ ID NO: 5, SEQ ID NO: 6, SEQ ID NO: 7, SEQ ID NO:8, SEQ ID NO: 9, SEQ ID NO: 10, or SEQ ID NO: 11.

15. The method of claim 14, wherein the antibody or antigen binding fragment comprises a heavy chain variable region and a light chain variable region, wherein the heavy chain variable region comprises the sequence of SEQ ID NO:3, and wherein the light chain variable region comprises a sequence selected from the group consisting of SEQ ID Nos: 5-8.

16. The method of claim 15, wherein the light chain variable region comprises the sequence of SEQ ID NO:8.

17. The method of claim 14, wherein the antibody or antigen binding fragment comprises a heavy chain variable region and a light chain variable region, wherein the heavy chain variable region comprises the sequence of SEQ ID NO:4, and wherein the light chain variable region comprises a sequence selected from the group consisting of SEQ ID Nos: 9-10.

18. The method of claim 1, wherein the antibody or antigen binding fragment comprises a heavy chain variable region and a light chain variable region, wherein the heavy chain variable region comprises a sequence having a 90-100% sequence identity to SEQ ID NO:3, and wherein the light chain variable region comprises a sequence having a 90-100% sequence identity to a sequence selected from the group consisting of SEQ ID Nos: 5-11.

19. The method of claim 1, wherein the subject is a human.
